(19) United States
(12) Patent Application Publication
(10) Pub. No.: US 2010/0121117 A1
(43) Pub. Date: May 13, 2010

(54) PROCESS FOR PREPARING DECAFLUOROCYCLOHEXENE

(75) Inventors: Hyang Ja JANG, Ulsan (KR); Jong Yool Yang, Ulsan (KR); Chul Ho Kim, Ulsan (KR); Young Gu Cho, Ulsan (KR); Jung Eun Lee, Ulsan (KR)

Correspondence Address: EGBERT LAW OFFICES, 412 MAIN STREET, 7TH FLOOR, HOUSTON, TX, 77002, US

(73) Assignee: FOOSUNG Co. Ltd., Ulsan (KR)

(21) Appl. No.: 12/388,876

(22) Filed: Feb. 19, 2009

(30) Foreign Application Priority Data
Nov. 13, 2008 (KR) 10-2008-0112622

(51) Int. Cl. C07C 17/04

(52) U.S. Cl. 570/153

(57) ABSTRACT

Disclosed herein is a process of preparing decafluorocyclohexene using hexafluorobenzene as a raw material. The hexafluorobenzene reacts with an activated fluorinating agent at 60-200° C. in an inert gas atmosphere. The activated fluorinating agent is prepared by mixing 1-50 wt % of cobalt difluoride with 50-99 wt % of other metal fluoride selected from calcium fluoride, magnesium fluoride, aluminum fluoride, sodium fluoride and potassium fluoride. The mixture reacts with fluorine gas at 200-400° C.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to a process of preparing decafluorocyclohexene by allowing hexafluorobenzene to react with a fluorinating agent, which yields decafluorocyclohexene with high selectivity, and to a fluorinating agent which is used in the process.

More specifically, the present invention relates to a process of preparing decafluorocyclohexene (C₆F₁₀) by allowing hexafluorobenzene (C₆F₆) to react with a fluorinating agent. The invention also relates to a fluorinating agent comprising 1-50 wt % of cobalt fluoride (CoF₂) and 50-99 wt % of other metal fluoride selected one at least among calcium fluoride (CaF₂), magnesium fluoride (MgF₂), aluminum fluoride (AlF₃) sodium fluoride (NaF) and potassium fluoride (KF).

2. Description of Related Art Including Information Disclosed Under 37 CFR 1.97 and 37 CFR 1.98

Decafluorocyclohexene (C₆F₁₀) is a compound having a molecular weight of 262 and a boiling point of 51-53° C. It's chemical species present in the plasma state is similar to octafluorocyclopentene (C₅F₈), an etching gas which is used for polysilicon or silicon oxide in the prior art.

The present inventors have considered that decafluorocyclohexene (C₆F₁₀) which can be commercially prepared through an easy and simple process could be a useful etching gas, and have developed a preparation process thereof.

GB Patent No. 920,796 discloses a process of preparing decafluorocyclohexene (C₆F₁₀) by heating undecafluorocyclohexane (C₆F₁₁H) at 700-900° C.

In this process, the reaction temperature has maintained at 700° C. at lowest, but the yield was as low as 61-85%. In GB Patent No. 1,017,814, decafluorocyclohexene (C₆F₁₀) was prepared with low yield bypassing undecafluorocyclohexane (C₆F₁₁H) through sodium fluoride pellets at a reaction temperature of 320° C. In addition, the preparation of undecafluorocyclohexane (C₆F₁₁H) as the raw material is carried out in a complicated manner, thus, the industrial application thereof is greatly limited.

U.S. Pat. No. 3,331,880 discloses a process of preparing decafluorocyclohexene (C₆F₁₀) by allowing an excess amount of antimony pentafluoride (SbF₅) to react with chloroperfluorocyclohexene at a temperature of 120° C. When heptafluorotrichlorocyclohexene is used as the raw material, the yield of decafluorocyclohexene (C₆F₁₀) is as extremely low as 7.1%.

In addition, a process is known for preparing decafluorocyclohexene (C₆F₁₀) by allowing undecafluorocyclohexane (C₆F₁₁H) to react with KOH (Fluorocarbon and their derivatives. R. E. bank, 1970).

### BRIEF SUMMARY OF THE INVENTION

It is an object of the present invention to provide a process of preparing high-purity decafluorocyclohexene in high yield by fluorinating hexafluorobenzene (C₆F₆) as a starting material and to provide a fluorinating agent which is used in the fluorination process.

The fluorinating agent developed in the present invention is a powder and is prepared by mechanically mixing 1-50 wt % of cobalt difluoride (CoF₂) with 50-99 wt % of other metal fluoride selected one at least among calcium fluoride (CaF₂), magnesium fluoride (MgF₂), aluminum(III) fluoride (AlF₃), sodium fluoride (NaF) and potassium fluoride (KF) and bringing the mixture into contact with fluorine gas, thus preparing an activated fluorinating agent. Reactivity and selectivity are vary depending on the ratio of metal fluoride added, and this fluorinating agent is suitable for selectively synthesizing decafluorocyclohexene (C₆F₁₀).

The activation for the fluorinating agent is carried out by allowing CoF₂ and the metal fluoride to react with fluorine gas so as to activate the metal fluoride, and it proceeds according to the following reaction equation:

CoF₂+½F₂→CoF₃

Metal fluorides other than CoF₂ no longer undergo a fluorination reaction during the process of activating the fluorinating agent.

CoF₃ is a good fluorinating agent but unstable compound which is reduced into CoF₂ immediately upon contact with air and is very difficult to store.

When C₆F₆ is used as a starting material to prepare C₆F₁₀, with CoF₃ as a fluorinating agent, the reaction product will be obtained as a mixture of C₆F₈, C₆F₁₀ and C₆F₁₂ in various ratios. Particularly if only CoF₃ is used, most of the reaction product will be C₆F₁₂.

Herein, the reaction of C₆F₆ is influenced by the fluorinating ability of the fluorinating agent and the reaction temperature, and the mixing ratio of components of the metal oxides.

Accordingly, in order to increase the selectivity of the target compound (C₆F₁₀), it is required to maintain reaction condition suitable therefor.

The present inventors have conducted various experiments to determine reaction condition in which the selectivity of the target compound (C₆F₁₀) can be maximized. As a result, the present inventors have found that the fluorination rate of C₆F₆ can be adjusted by adjusting the weight ratio of CoF₃ in the fluorinating agent, and have found reaction conditions, in which the selectivity of C₆F₁₀ is exceptionally high, by selecting and employing a metal fluoride as a diluting agent to adjust the content ratio of CoF₃ without adversely affecting a fluorination reaction, thereby completing the present invention.

Herein, the metal fluoride is preferably CaF₂, MgF₂, AlF₃, NaF or KF.

In order to inhibit C₆F₆ from proceeding to C₆F₁₂ due to the high activity of CoF₃ and to control reaction conditions, the activity of CoF₃ needs to be adjusted to a low level.

The process of preparing decafluorocyclohexene using the activated fluorinating agent is characterized in that, as a method of adjusting the reactivity of the fluorinating agent in order to obtain the optimal yield, in addition to adjusting the ratio of metal fluoride added, any one of nitrogen (N₂), helium (He) and argon (Ar) that are inert gases is supplied together with the reactant.

When inert gas is introduced into the fluorination reaction of C₆F₆, the contact time between the raw material gas (C₆F₆) and the fluorinating agent can be reduced. Thus, the fluorination reaction can be controlled through the introduction of inert gas.

The fluorinating agent comprises CoF₂ as an active ingredient and metal fluoride as a diluen, and the content of the metal fluoride is preferably 50-99 wt % based on 100 wt % of the fluorinating agent.

Under the conditions of a fluorination reaction that uses the fluorinating agent of the present invention, the content of metal fluoride, the reaction temperature and the amount of inert gas supplied influence the fluorination reaction. If the content of metal fluoride is more than 99%, the fluorination rate will be excessively low, and if it is less than 50 wt %, the control of the reaction will be difficult.

The content of metal fluoride is preferable for 50-90 wt % to control the reaction rate at the reaction temperature of 60˜200° C.

In the preparation process of the present invention, hexafluorobenzene is used as a starting material. Hexafluorobenzene (C₆F₆) is a compound which contains a fluorine atom bonded to each of six unsaturated carbons, has no hydrogen, and is liquid at room temperature. The use of benzene, chlorobenzene or a hydrocarbon as a starting material is not suitable not only because Hydrogen fluoride(HF) is produced, which needs additional process of removing acid, but also because many side products including isomers are produced, thus making a purification process very complicated.

In the present invention, a mixture of cobalt difluoride (CoF₂) and metal fluoride is charged into a reactor and activated with fluorine gas at 200-400° C., thus preparing an activated fluorinating agent. C₆F₆ together with inert gas is supplied and allowed to react with the activated fluorinating agent at a reaction temperature of 60-200° C., thus preparing C₆F₁₀. After completion of the reaction, the fluorinating agent is activated again with fluorine gas and stands by for the next reaction. For commercial application, the reaction must be continuously carried out, and thus two reactors are used such that the process of activating the fluorinating agent and the reaction process are repeatedly carried out.

Hereinafter, the preparation process of the present invention will be described with reference to the preparation system shown in FIG. 1.

### BRIEF DESCRIPTION OF THE SEVERAL VIEWS OF THE DRAWINGS

The above and other objects, features and advantages of the present invention will be more clearly understood from the following detailed description taken in conjunction with the accompanying drawing.

FIG. 1 is a schematic view of a diagram showing a process of preparing decafluorocyclohexene according to the present invention.

### DETAILED DESCRIPTION OF THE INVENTION

### Type of Reactor and Process for Activating Fluorinating Agent

In a horizontal-type or vertical-type reactor, a fluorinating agent obtained by mechanically mixing 1-50 wt % of powdery cobalt difluoride (CoF₂) with 50-99 wt % of at least one of powdery calcium fluoride (CaF₂), magnesium fluoride (MgF₂) aluminum fluoride (AlF₃), sodium fluoride (NaF) and potassium fluoride (KF) is uniformly charged in an amount corresponding to about 70% of the volume of the reactor and activated with fluorine gas at a temperature of 200-400° C. After completion of the activation, the remaining fluorine gas is removed while supplying inert gas into the reactor. When the introduced fluorine gas is no longer consumed in the reactor the activation of CoF₂ is ended.

Process for Synthesizing C₆F₁₀

After the activation of the fluorinating agent is completed, the temperature of the reactor is lowered to 60-200° C., and the raw material C₆F₆ is quantitatively passed through a preheater, and then sent to the reactor. The reaction product from the reactor is condensed in a trap at a temperature ranging from −10° C. to 0° C. to capture unreacted reactant and products having higher boiling points, and the remaining product is condensed in a second trap of acetone/liquid nitrogen slush at a temperature ranging from −60° C. to −80° C.

Hereinafter, the present invention will be described in further detail with reference to examples.

### EXAMPLES 1 TO 4

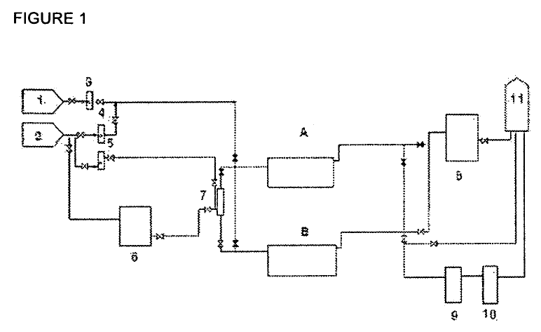

As shown in FIG. 1, 1.8 kg of a fluorinating agent consisting of 66.7 wt % of MgF₂ and 33.3 wt % of CoF₂ was introduced in a reactor A or B (3 inches×1100 mm). Then, the inside of the reactor was purged with nitrogen gas to remove water from the reactor and the powder surface. Then, the temperature of the reactor was elevated to 350° C., and fluorine gas was introduced therein to activate the fluorinating agent. After completion of the activation, unreacted fluorine gas in the reactor was removed, and the temperature of the reactor was maintained at 70-130° C.

Then, the raw material C₆F₆ was quantitatively passed through a preheater 7 at a temperature of 120-130° C. and sent to the reactor. In addition, nitrogen gas was also supplied into the reactor in an amount of 5-600 mol % relative to the amount of raw material supplied.

The reaction of hexafluorobenzene (C₆F₆) with the fluorinating agent prepared according to the above-described method was carried out under the following reaction conditions.

Reaction Conditions:
Reactor: 3 inches×1100 mm, SUS 316L
Raw material: hexafluorobenzene (70-90 g/hr)
Supply ratio of inert gas: 180 mol % relative to the amount of raw material supplied (inert gas: nitrogen)
Reaction temperature: 70-130° C.
Reaction pressure: atmospheric pressure

After the hexafluorobenzene was fluorinated under the above-described conditions, the resulting gas was condensed using both trap of ice water and trap of acetone/liquid nitrogen slush. Then, the reaction product was analyzed using gas chromatography. The analysis results are shown in Tables 1 and 2 below.

### EXAMPLES 5 TO 9

Examples 5 to 9 were carried out according to the reaction conditions shown in Tables 1 and 2 in the same manner as in Examples 1 to 4, except that the compositions and contents of the fluorinating agent were changed. The results are shown in Tables 1 and 2 below.

TABLE 1

| | Examples | | | | | | | | | | | |
|---|---|---|---|---|---|---|---|---|---|---|---|---|
| | 1 | | 2 | | 3 | | 4 | | 5 | | 6 | |
| Fluorinating | 33.3% CoF₂/MgF₂ | | | | 10% CoF₂/MgF₂ | | | | 3% CoF₂/MgF₂ | | | |
| agent | | | | | | | | | | | | |
| Reaction | 115 | | 105 | | 95 | | 70 | | 105 | | 105 | |
| temp° C. | | | | | | | | | | | | |
| Inert gas | 10 | | 100 | | 100 | | 100 | | 100 | | 100 | |
| (ml/min) | | | | | | | | | | | | |
| Contact | 380 | | 180 | | 210 | | 180 | | 180 | | 180 | |
| time | | | | | | | | | | | | |
| (sec) | | | | | | | | | | | | |
| | A | B | A | B | A | B | A | B | A | B | A | B |
| C₆F₆ | 100 | 100 | 100 | 100 | 100 | 100 | 97.27 | 99.1 | 66.2 | 84.4 | 29.6 | 49.1 |
| conv | | | | | | | | | | | | |
| (%) | | | | | | | | | | | | |

Composition (mol %) of organic compounds

| | | | | | | | | | | | | |
|---|---|---|---|---|---|---|---|---|---|---|---|---|
| C₆F₁₂ | 11.30 | 24.0 | 0.96 | 33.30 | 0.96 | 1.43 | 0.04 | 0.06 | 0.06 | 0.12 | 0.17 | 0.51 |
| C₆F₁₀ | 86.90 | 74.20 | 98.91 | 62.00 | 98.91 | 98.47 | 96.34 | 97.37 | 85.14 | 84.05 | 69.18 | 77.25 |
| C₆F₈ | 1.60 | 0.70 | 3.60 | 1.30 | 0.01 | 0.0 | 3.27 | 2.30 | 14.31 | 15.24 | 29.00 | 21.70 |

TABLE 2

| | Examples | | | | | |
|---|---|---|---|---|---|---|
| | 7 | | 8 | | 9 | |
| Fluorinating agent | 33.3% CoF₂/MgF₂ | | | | | |
| Reaction temperature (° C.) | 112 | | 110 | | 50 | |
| Inert gas (ml/min) | 10 | | 10 | | 100 | |
| Contact time (sec) | 380 | | 180 | | 210 | |
| | A | B | A | B | A | B |
| C₆F₆ conversion (%) | 100 | 100 | 99.7 | 99.7 | 96.5 | 99.1 |

Composition (mol %) of organic compounds

| | | | | | | |
|---|---|---|---|---|---|---|
| C₆F₁₂ | 8.3 | 10.2 | 16.4 | 21.5 | 0.19 | 1.43 |
| C₆F₁₀ | 91.2 | 89.4 | 83.0 | 77.7 | 10.01 | 98.47 |
| C₆F₈ | 0.5 | 0.5 | 0.6 | 0.4 | 8.87 | 0.01 |

In the results shown in Tables 1 and 2 above, as the ratio of CoF₃ had increased, the conversion of C₆F₆ was increased, but the selectivity of C₆F₁₀ was decreased. Under optimal reaction condition, the selectivity of C₆F₁₀ could reach about 99%.

In the present invention, the two reactors A and B are connected in parallel and used alternately. Specifically, when the reaction in one reactor is completed, the remaining organic product is discharged with nitrogen, and then fluorine gas is introduced into the reactor to activate the fluorinating agent. At the same time, in the other reactor which is standing by, the reaction of the organic material with the fluorinating agent is initiated. For commercial application, the reaction must be continuously carried out, and thus two reactors are alternatively used in order for the process of activating the fluorinating agent and the reaction process to be repeatedly carried out.

As described above, the process of the present invention has an advantage in that decafluorocyclohexene can be prepared with a selectivity of more than 99%.

Although the preferred embodiment of the present invention has been described for illustrative purposes, those skilled in the art will appreciate that various modifications, additions and substitutions are possible, without departing from the scope and spirit of the invention as disclosed in the accompanying claims.

1-2. (canceled)

3. A process of preparing decafluorocyclohexene using hexafluorobenzene as a raw material, the process comprising the steps of:
reacting hexafluorobenzene with an activated fluorinating agent at 60-200° C. in an inert gas atmosphere so as to achieve a high yield of C₆F₁₀;
preparing said activated fluorinating agent by mixing 1-50 wt % of cobalt difluoride with 50-99 wt % of other metal fluoride selected from a group consisting of calcium fluoride, magnesium fluoride, aluminum fluoride, sodium fluoride and potassium fluoride to prepare a mixture; and
reacting the mixture with fluorine gas at 200-400° C.

4. The process of claim 3, wherein the inert gas is selected from the group consisting of nitrogen, helium and argon, the inert gas being supplied in an amount of 50-400 mol % relative to the amount of hexafluorobenzene supplied.
